(19) United States
(12) Patent Application Publication
(10) Pub. No.: US 2012/0219993 A1
(43) Pub. Date: Aug. 30, 2012

(54) METHOD OF PRODUCING MICROBIAL INTRACELLULAR PRODUCTS FROM VOLATILE FATTY ACIDS

(75) Inventors: Ho Nam CHANG, Daejeon (KR); Qiang Fei, Daejeon (KR); Jin Dal Rae Choi, Busan (KR); Nag Jong Kim, Daejeon (KR); Woohyun Kim, Woolsan (KR)

(73) Assignee: KOREA ADVANCED INSTITUTE OF SCIENCE AND TECHNOLOGY, Daejeon (KR)

(21) Appl. No.: 13/299,274

(22) Filed: Nov. 17, 2011

(30) Foreign Application Priority Data
Feb. 28, 2011 (KR) 10-2011-0018098

(51) Int. Cl. C12P 21/00; C12P 7/64; C12P 1/02; C12P 1/00; C12P 1/04

(52) U.S. Cl. 435/71.1; 435/41; 435/170; 435/171; 435/134

(57) ABSTRACT

A method for producing microbial intracellular products, and more particularly a method of producing microbial intracellular products from volatile fatty acids derived from organic waste is provided. The method of producing microbial intracellular products from volatile fatty acids in a multi-stage continuous high-cell-density culture bioreactor system includes the steps of: (a) culturing microorganisms in a bioreactor for microbial growth, thereby growing the microorganisms; (b) culturing the grown microorganisms of step (a) in a bioreactor for production of microbial intracellular products, which includes a medium containing volatile fatty acids, thereby producing microbial intracellular products; and (c) recovering the intracellular microbial products from the culture medium of the bioreactor for production of microbial intracellular products.

### CROSS-REFERENCE TO RELATED APPLICATIONS

This application claims the benefit of Korean Patent Application No. 10-2011-0018098, filed on Feb. 28, 2011 in the Korean Intellectual Property Office, which is incorporated herein by reference in its entirety.

### BACKGROUND OF THE INVENTION

1. Field of the Invention

The present invention relates to a method for producing microbial intracellular products, and more particularly to a method of producing microbial intracellular products from volatile fatty acids derived from organic waste.

2. Background of the Related Art

In general, biodiesel is produced from vegetable oils such as soybean oil, palm oil, rapeseed oil, etc. Because the production of vegetable oils depends on the plant growth area and climate, vegetable oils should be produced in a specific area and cannot be produced throughout the year. Also, because a large area of land is required for plant growth, the production of vegetable oils in a high-land-price area is cost-ineffective and even palm oil whose production is the highest among vegetable oils is produced with a low productivity of 5 tons per hectare. For this reason, about 80% of the production cost of biodiesel corresponds to the raw material cost. Also, for this reason, it is difficult to spread the use of biodiesel, even though biodiesel is completely compatible with the existing transportation fuel (diesel).

It is known that microorganisms grow at a significantly higher rate than plants and some microorganisms have a high lipid content of 20-80%. Among microorganisms, yeasts and microalgae have been most actively studied, and yeasts known to be involved in the production of lipids from sugars such as glucose include Candida (Evans, C. T. and Ratledge, Lipids, 18: 623-629, 1983), Lipomyces (Naganuma, T. et al., J. Gen. Appl. Microbiol., 31: 29-37, 1985), Rhodotorula (Yoon, S., et al., J. Ferment. Technol., 60: 243-246, 1982), and Cryptococcus (Fall, R. et al., Appl. Environ. Mircobiol., 47: 1130-1134, 1984).

Also, it was reported that the microalga Chlorella uses carbon dioxide or glucose for lipid production and that the microalga Botryococcus uses carbon dioxide for lipid production.

However, glucose sources are mostly corn, cassava, and the like, which are used as human foods, and sugar is produced only in specific areas and widely used as food additives, and thus the use thereof as a biofuel source is limited. Also, lignocellulosic biomass-derived glucose, xylose and the like can be used as substrates for production of microbial lipids, but there is a problem in that a saccharification process for obtaining them requires much energy and cost.

Meanwhile, volatile fatty acids are acidic organic compounds that mainly contain a carboxyl group and a sulfone group. Such volatile fatty acids can be readily produced from organic waste, including proteins, fats and carbohydrates, and can also be produced by anaerobic digestion of lignocellulosic biomass in an inexpensive manner. However, volatile fatty acids have not yet received attention as a substrate for microbial culture, unlike sugars or alcohols. This is because microorganisms that effectively use volatile fatty acids are rare and volatile fatty acids themselves inhibit the growth of most microorganisms.

The concentration of microbial cells in a bioreactor together with the activity of microbial cells plays an important role in producing various useful substances (ethanol, lactic acid, volatile fatty acid, penicillin, monoclonal antibody, various proteins, etc.) by microbial fermentation. Microbial fermentation products are largely divided into extracellular products, including ethanol, lactic acid, penicillin and monoclonal antibody, which are released from microbial cells, and intracellular products that are produced in microbial cells. Typical examples of intracellular products include proteins, PHB (polyhydroxybutryrate), microbial lipids and the like which are produced in genetic recombinant E. coli. The most important factors in biological processes that use microorganisms are the concentration and productivity of fermentation products. The present inventors increased ethanol production by more than 30 times by operating a bioreactor at a cell concentration of 200 g/L using a hollow fiber cell recycle system (Lee, C W and Chang, H N, Biotechnol Bioeng, 29, 1105-1112, 1987), but found a problem in that ethanol concentration in the product is 70% of that in a batch reactor. In an attempt to solve this problem, the present inventors succeeded in obtaining a product at high concentration in high yield by operating several high-cell-density reactors connected in series with each other (US 20100041124A1).

However, the strategy of application of multi-stage continuous high-cell-density culture (MSC-HCDC) is different between microbial extracellular products (e.g., ethanol) and microbial intracellular products (e.g., microbial lipids). The strategy of maintaining high concentration is equal between the extracellular products and the intracellular products, but the production of the extracellular products is performed so that the use of a substrate for the production of microbial cells is minimized while the use of the substrate for the production of the extracellular products is maximized, and the production of the intracellular products is performed so that the production of microbial cells can be maximized and the accumulation of microbial products such as lipids in the second and third reactors is maximized. The production of the extracellular products can also be performed using several reactors, if necessary, but the intracellular products such as microbial lipids can be produced with high productivity using 2 or 3 high-cell-density reactors connected in series to each other.

Accordingly, the present inventors have made extensive efforts to solve the above-described problems and, as a result, when the growth of microorganisms either in a single bioreactor or in a microbial growth bioreactor of a multi-stage continuous high-cell-density bioreactor system is maximized and then the microorganisms are cultured in a single bioreactor or in a bioreactor for production of microbial intracellular products, sugars such as glucose can be used as carbon sources in the single bioreactor or the bioreactor for production of microbial intracellular products, and the inhibition of microbial growth can be minimized and the production of microbial intracellular products can be maximized, even if volatile fatty acids produced from organic waste or inexpensive organic materials are used, thereby completing the present invention.

### SUMMARY OF THE INVENTION

Accordingly, it is an object of the present invention to provide a method of industrially producing microbial intracellular products in a cost-effective manner, in which the method can use sugars such as glucose, uses inexpensive volatile fatty acids as substrates (carbon sources) while minimizing microbial growth inhibition resulting from the volatile fatty acids, maximizes the production of microbial intracellular products, and maximizes the productivity of a microbial reactor using a single bioreactor or a multi-stage continuous high-cell-density reactor system.

To achieve the above object, the present invention provides a method for producing microbial intracellular products, the method including growing microorganisms in a bioreactor and subjecting the grown microorganisms to fed-batch culture using volatile fatty acids as substrates, thereby producing microbial intracellular products.

The present invention also provides a method of producing microbial intracellular products from volatile fatty acids in a multi-stage continuous high-cell-density bioreactor system, the method including the steps of: (a) culturing microorganisms in a bioreactor for microbial growth, thereby growing the microorganisms; (b) culturing the grown microorganisms of step (a) in a bioreactor for production of microbial intracellular products, which includes a medium containing volatile fatty acids, thereby producing microbial intracellular products; and (c) recovering the microbial intracellular products from the culture medium of the bioreactor for production of microbial intracellular products.

According to the present invention, microbial intracellular products can be produced in a cost-effective manner using inexpensive volatile fatty acids as carbon sources instead of glucose. Among the produced microbial intracellular products, microbial lipids can be used as a raw material for biodiesel, and thus can also contribute to increasing the cost-effectiveness of biodiesel.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 schematically shows a method of producing microbial intracellular products from volatile fatty acids in a multi-stage continuous high-cell-density bioreactor system according to one embodiment of the present invention.

FIG. 2 is a set of graphs showing curves of growth of Rhodococcus opacus in media containing volatile fatty acids according to one embodiment of the present invention.

FIG. 3 is a graph showing the change in lipid content in Cryptococcus albidus as a function of the C/N ratio according to one embodiment of the present invention.

FIG. 4 shows multi-stage continuous high-cell-density bioreactor system 2 according to one embodiment of the present invention.

### DETAILED DESCRIPTION OF THE PREFERRED EMBODIMENTS

Now, a preferred embodiment of the present invention will be descried hereinafter in more detail with reference to the accompanying drawings.

The present inventors have predicted that, when microorganisms are first grown and then cultured in a medium containing volatile fatty acids, the volatile fatty acids that are generally known to inhibit microbial growth can be advantageously used as carbon sources for production of microbial intracellular products.

In the present invention, microorganisms were cultured in a medium containing little or no volatile fatty acids. As a result, it was shown that the production of microbial lipid among microbial intracellular products was increased.

Specifically, in one Example of the present invention, microorganisms were grown in a bioreactor including a nutrient medium, and then cultured while a medium containing volatile fatty acids was added thereto. As a result, it could be seen that the production of microbial lipids was increased.

Accordingly, in one aspect, the present invention is directed to a method for producing microbial intracellular products, the method including growing microorganisms in a bioreactor and subjecting the grown microorganisms to fed-batch culture using volatile fatty acids as substrates, thereby producing microbial intracellular products.

The bioreactor that is used in the present invention may be a conventional bioreactor, and the microorganisms that are used in the present invention may be bacteria, yeasts, fungi or microalgae, which have the ability to produce intracellular products.

Examples of bacteria that may be used in the present invention include, but are not limited to, Acinetobacter, Actinobacter, Anabaena, Arthrobacter, Bacillus, Clostridium, Flexibacterium, Micrococcus, Mycobacterium, Nocardia, Nostoc, Oscillatoria, Pseudomonas, Rhodococcus, Rhodomicrobium, Rhodopseudomonas, Shewanella, Streptomyces, Vibrio, and the like. Examples of yeasts that may be used in the present invention include, but are not limited to, Lipomyces, Trichosporon, Rhorosporidium, Cryptococcus, Candida, Yarrowia, and the like. Examples of fungi that may be used in the present invention include, but are not limited to, Aspergillus, Chaetomium, Clodosporidium, Cunninghamella, Emericella, Fusarium, Mortierella, Mucor, Penicillium, Pythium, Rhizopus, Trichoderma, and the like, and examples of microalgae that may be used in the present invention include, but are not limited to, Botryococcus, Brachiomonas, Chlamydomonas, Chlorella, Crypthecodinium, Dunaliella, Euglena, Nannochloris, Nannochloropsis, Navicula, Nitzschia, Schizochytrium, Sceletonema, Scenedesmus, Tetraselmis, Thraustochytrium, Ulkenia, and the like.

In the present invention, the microbial intracellular products are preferably selected from the group consisting of intracellular proteins, intracellular organic polymers, and microbial lipids.

Examples of the microbial intracellular lipids include oleic acid, palmitic acid, stearic acid, linoleic acid, linolenic acid, etc.

Examples of the intracellular proteins include growth hormone, insulin, penicillin acylase, hepatitis vaccine, etc., and examples of the intracellular organic polymers include polyhydroxybutyric acid, poly-gamma-glutamic acid, polylactic acid, polyamino acid and the like.

The medium for microbial growth preferably does not contain volatile fatty acids as carbon sources or contains volatile fatty acids in an amount that does not influence microbial growth.

In another Example of the present invention, the yeast Cryptococcus albidus ATCC10672 having the ability to produce lipids was cultured and grown in a bioreactor for microbial growth including a medium containing glucose or sugar as a carbon source, and was then cultured in a bioreactor for production of microbial intracellular products, which includes a medium containing volatile fatty acids. As a result, it could be seen that the production of microbial lipids was increased.

Accordingly, in another aspect, the present invention is also directed to a method of producing microbial intracellular products from volatile fatty acids or sugars such as glucose in a multi-stage continuous high-cell-density bioreactor system, the method including the steps of: (a) culturing microorganisms in a bioreactor for microbial growth, thereby growing the microorganisms; (b) culturing the grown microorganisms of step (a) in a bioreactor for production of microbial intracellular products, which includes a medium containing volatile fatty acids, thereby producing microbial intracellular products; and (c) recovering the microbial intracellular products from the culture medium of the bioreactor for production of microbial intracellular products.

In the present invention, as shown in FIG. 1, in order to produce microbial intracellular products using volatile fatty acids, a step (S1) of culturing microorganisms in a bioreactor for microbial growth to grow the microorganisms is carried out.

The volatile fatty acids are as defined above.

Because the step of culturing microorganisms in the bioreactor for microbial growth aims to grow the microorganisms, the medium for microbial growth preferably does not contain volatile fatty acids as carbon sources or contains volatile fatty acids in an amount that does not influence microbial growth. The carbon/nitrogen (C/N) ratio of the medium influences the growth rate of cells and the production rate and content of products and can slightly vary depending on the kind (bacteria, yeasts and fungi etc.) and growth time of microorganisms, but the carbon/nitrogen (C/N) ratio is preferably 2-20. If the carbon/nitrogen (C/N) ratio is less than 2, nitrogen will not be completely consumed due to excessively high nitrogen concentration, thus increasing costs and environmental problems, and if the C/N ratio is more than 20, the carbon source will not be completely consumed and will be discarded, thus increasing costs.

Specifically, the microorganisms in the bioreactor for microbial growth are preferably grown in a medium containing (1) a carbon (C) source selected from the group consisting of glucose, xylose, arabinose, sugar, alcohol, sugar alcohol, a saccharification product of biomass, and mixtures thereof, and (2) a nitrogen (N) source selected from the group consisting of ammonia-derived compounds, nitric oxide, urea, corn steep liquor, food waste, and mixtures thereof.

Examples of the saccharification product of biomass include saccharification products of woody and grassy biomass, seaweeds and aquatic plants, and examples of the ammonia-derived compounds include ammonium chloride (NH₄Cl), ammonia water (NH₄OH), ammonium carbonate (e.g., NH₄HCO₃, (NH₄)₂CO₃) or commercially available corn steep liquor (CSL), and examples of the nitric oxide include compounds (e.g., (HNO₃, KNO₃) containing nitrate nitrogen (NO₂, NO₃).

The carbon/nitrogen (C/N) ratio of the medium can be controlled by changing the content of nitrogen relative to the content of carbon, or vice versa. The content of nitrogen can be determined by measuring the total nitrogen amount and the amount of carbon in total carbon sources, but is preferably determined based on dioses such as glucose and sugar, which are easily decomposed.

In the present invention, each of the bioreactor for microbial growth and the bioreactor for production of microbial intracellular products preferably consists of a reactor for multi-stage continuous high-cell-density culture (MSC-HCDC). The bioreactor for microbial growth is preferably operated in the form of a continuous stirred tank reactor (CSTR), and the bioreactor for production of microbial intracellular products is operated in the form of several CSTRs or is operated in the form of a plug flow reactor (PFR) provided by dividing the inside of one CSTR into several sections.

In the present invention, the microorganisms having the ability to produce intracellular products can be cultured by shake culture, stationary culture, batch culture, fed-batch culture, continuous culture, or the like.

As used herein, the term “shake culture” means a method in which microorganisms are cultured while the medium inoculated with the microorganisms is shaken, and the term “stationary culture” means a method in which microorganisms are cultured while the medium inoculated with the microorganisms is not shaken. Also, the term “batch culture” means a method in which microorganisms are cultured in a state in which the volume of the medium was fixed without adding a fresh medium, and the term “fed-batch culture” has a meaning contrary to a batch culture process of adding the entire amount of a raw material to a culture tank at the initial stage, followed by culture, and means a method in which small amounts of elements are added to a culture tank after which a raw material is added thereto little by little during the culture process. In addition, the term “continuous culture” means a culture method in which a fresh nutrient medium is continuously fed while a culture medium containing cells and products is continuously removed.

The culture conditions, including culture time, oxygen concentration, pH and temperature, can be suitably controlled depending on the kind of microorganisms used.

The step (S1) is followed by a step (S2) of culturing the grown microorganisms in a bioreactor for production of microbial intracellular products, which include a medium containing volatile fatty acids, to produce microbial intracellular products.

Because the step of culturing the microorganisms in the bioreactor for production of microbial intracellular products aims to product microbial intracellular products using the medium containing volatile fatty acid, the medium preferably contains volatile fatty acids as carbon sources. The carbon/nitrogen (C/N) ratio of the medium influences the degree of accumulation of microbial lipids and the growth rate of microorganisms and is preferably 10 or more, although it can vary depending on the kind of microorganisms used. If the carbon/nitrogen (C/N) ratio is less than 10, the content of microbial intracellular products will be low so that the cost for recovering the intracellular products will increase. Usually, the concentration of the carbon sources in the medium does not exceed 200 g/L that is a concentration to which microorganisms can use the carbon sources.

The volatile fatty acids can be obtained by anaerobically fermenting organic waste or an inexpensive organic material to produce 10-50 g/L of volatile fatty acids before carrying out microbial culture in the bioreactor for microbial growth or the bioreactor for production of microbial intracellular products, and the obtained volatile fatty acids can be used directly without special pretreatment or can be used after sterilization.

Examples of the organic waste include urban waste, food waste, sludge, livestock excretions, organic biomass, and the like, and examples of the inexpensive organic material include grassy or woody (lignocellulosic) biomass, seaweeds, microalgae, and the like.

Examples of the volatile fatty acids include, but are not limited to, acetic acid, propionic acid, butyric acid, lactic acid, succinic acid, gluconic acid, valeric acid, caproic acid, and mixtures thereof.

It was reported that the volatile fatty acids are produced at a concentration of about 30˜50 g/L by fermentation of organic waste and have a volatile fatty acid ratio of 6 (acetic acid):1 (propionic acid):3 (butyric acid) (Sung-Jin Im et al, Bioresource Tech, 99, 7866-7874, 2008).

The volatile fatty acid-containing medium which is included in the bioreactor for production of microbial intracellular products can contain, in addition to the volatile fatty acids, (1) a carbon (C) source selected from the group consisting of glucose, xylose, arabinose, sugar, alcohol, sugar alcohol, a saccharification product of biomass, and mixtures thereof, and (2) a nitrogen (N) source consisting of ammonia-derived compounds, nitric oxides, urea, corn steep liquor, food waste, and mixtures thereof.

The medium containing the volatile fatty acids is preferably maintained at a volatile fatty acid concentration of 1-5 g/L during culture. If the content of the volatile fatty acids in the medium is less than 1 g/L, the effect of substituting glucose as a carbon source will be insufficient, and if the content is more than 5 g/L, it can inhibit microbial growth. For reference, in the case of microorganisms accumulating intracellular proteins, sugar is used because volatile fatty acids may be unable to use, and in the case of high-value-added compounds that are produced in small amounts, the price of the carbon sources can have an insignificant effect on the production cost.

The culture of the microorganisms in the bioreactor for production of microbial intracellular products can also be performed by shake culture, stationary culture, batch culture, fed-batch culture, continuous culture or the like. Also, culture conditions, including culture time, oxygen concentration, pH and temperature, can be suitably controlled depending on the kind of microorganisms used.

After completion of the culture of the microorganisms in the bioreactor for production of microbial intracellular products, a step (S3) of recovering the microbial intracellular products from the culture medium of the bioreactor for production of microbial intracellular products is carried out.

Recovery of the microbial intracellular products from the culture medium can be performed using filtration, centrifugation, precipitation, aggregation, adsorption or the like.

Specifically, if the microbial intracellular products are released from the microbial cells, these products can be separated from the filtrate by centrifugation or extraction, and if these products are accumulated in the microorganisms, these products can be extracted with chloroform, hexane, methanol, ethanol, dichloromethane, acetone, petroleum ether, or a mixture of two or more thereof, as generally known in the art, in which examples of the mixture include a mixture of hexane and methanol (2:1) and a mixture of chloroform and acetone (2:1).

If the extraction process is carried out using a hydrophobic solvent such as hexane, neutral lipids such as diacylglyceride and triacylglyceride can be extracted, and if the extraction process is carried out using a hydrophilic solvent such as ethanol, phospholipids can be extracted.

Intracellular produces such as intracellular proteins and polymers can be extracted into a solution by disrupting the microbial cell walls. The intracellular products extracted into the solution can be easily separated from the solution by a conventional method well known in the art.

In the present invention, the remaining microorganisms after recovering the microbial intracellular products from the culture medium of the bioreactor for production of microbial intracellular products can be used as a microbial strain for production of volatile fatty acids. Specifically, the remaining microorganisms after recovering the microbial intracellular products can be used as microorganisms for anaerobically digesting organic waste, such as sludge, food waste or excretions, grassy or woody (lignocellulosic) biomass, seaweed, microalgae and the like to produce volatile fatty acids.

### EXAMPLES

Hereinafter, the present invention will be described in further detail with reference to examples. It will be obvious to those skilled in the art that these examples are illustrative purposes only and are not to be construed to limit the scope of the present invention.

### Example 1

### Lipid Production by Microorganism Cryptococcus Albidus at Various Compositions of Mixed Volatile Fatty Acids

The effect of the ratio of acetic acid, propionic acid and butyric acid in volatile fatty acids (VFAs) on lipid production was examined, and the results are shown in Table 1 below. For this purpose, Cryptococcus albidus (ATCC 10672) was added at a concentration of 10% to a 250-ml flask containing 50 ml of modified basal medium (per liter; KH₂PO₄ (3.0 g); MgSO₄.7H₂O (1.0 g); FeCl₃.6H₂O (15 mg) and ZnSO₄.7H₂O (7.5 mg)) and was cultured at 150 rpm for 96 hours under conditions of 25° C. and pH of 6.0. The initial concentration of VFAs was set at 2 g/L, and experiments were carried out at various VFA compositions. As a result, it was found that the best blend ratio of acetic acid, propionic acid and butyric acid was 8:1:1.

TABLE 1

| Carbon | Biomass | Lipid | Lipid content | | |
|---|---|---|---|---|---|
| Source | (g/L) | (g/L) | % (w/w) | Y_{X/S}(g/g)^{a} | Y_{L/S}(g/g)^{b} |
| VFAs (4:3:3) | 0.642 | 0.127 | 19.8 | 0.321 | 0.063 |
| VFAs (8:1:1) | 1.201 | 0.334 | 27.8 | 0.601 | 0.167 |
| VFAs (6:1:3) | 1.168 | 0.319 | 27.3 | 0.584 | 0.159 |
| VFAs (7:2:1) | 1.115 | 0.291 | 26.1 | 0.558 | 0.146 |

Y_{X/S}: growth yield coefficient, g DCW/g VFAs.
Y_{L/S}: Lipid yield coefficient, g lipid/g VFAs.

### Example 2

### Microbial Lipid Production at Various VFA Contents

Lipid production by C. albidus (ATCC10672) was carried out under the same conditions as those of Example 1 using a medium containing glucose and a mixture of volatile fatty acids (acetic acid:propionic acid:butyric acid=6:1:3). The results are shown in Table 2 below.

TABLE 2

| | Biomass | Lipid | Lipid content | | |
|---|---|---|---|---|---|
| Carbon Source | (g/L) | (g/L) | % (w/w) | Y_{X/S}(g/g) | Y_{L/S}(g/g) |
| Glucose (18 g/L) | 8.402 | 3.3 | 39.3 | 0.467 | 0.183 |
| VFAs (2 g/L) | 1.155 | 0.312 | 27.0 | 0.578 | 0.156 |
| VFAs (5 g/L) | 2.554 | 0.635 | 24.9 | 0.511 | 0.127 |
| VFAs (8 g/L) | 0.784 | 0.093 | 11.9 | 0.261 | 0.031 |
| VFAs (10 g/L) | N.D.^{c} | N.D. | N.D. | N.D. | N.D. |

^{c}N.D.: not detected.

As shown in Table 2 above, when the concentration of volatile fatty acids was 2 g/L, the highest lipid production was shown, and when the initial concentration of volatile fatty acids was 10 g/L, microbial growth or lipid formation was not observed.

### Example 3

### Production of Microbial Lipids from Volatile Fatty Acids Using Bacteria

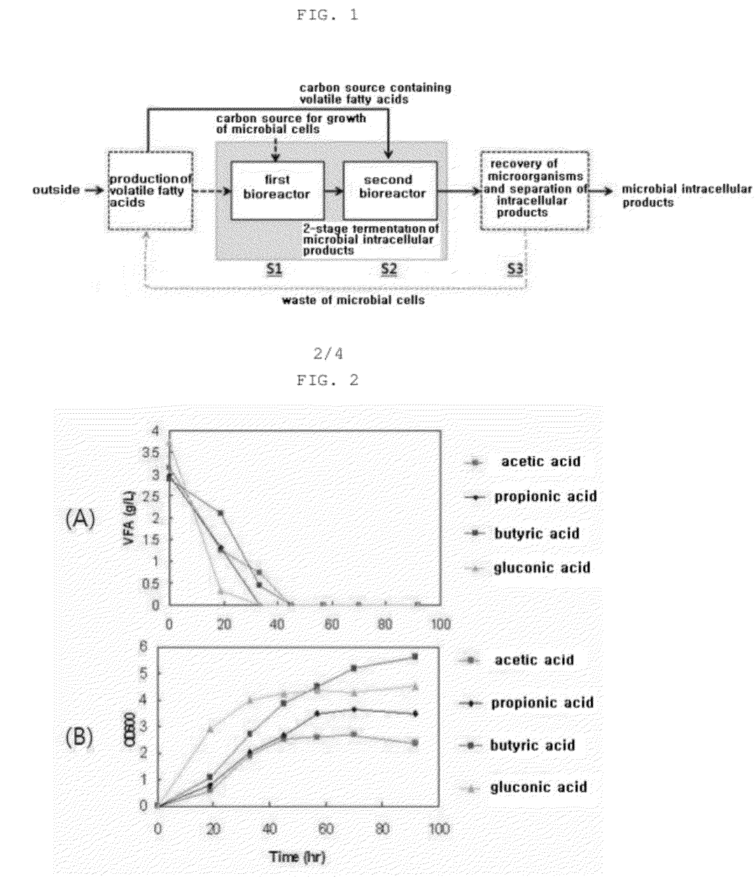

Lipid productivity from volatile fatty acids was carried out using the bacteria Rhodococcus opacus PD630 (DSM 44193). To prepare a culture medium, 3 g/L of each of volatile fatty acids, including acetic acid, butyric acid, propionic acid and gluconic acid, was added to MSM minimal medium (containing 9 g/L Na₂HPO₄.12H₂O, 1.5 g/L KH₂PO₄, 0.05 g/L NH₄Cl, 0.2 g/L MgSO₄.7H₂O, 0.5 g NaHCO₃, 20 mg CaCl₂.2H₂O, and 2 mL microelements) which was then adjusted to a pH of 6.5. R. opacus that has been cultured in nutrient broth for hours was inoculated into the minimal medium at a concentration of 5% and shake-cultured. As a result, as can be seen in FIG. 2B, the bacterial cells were grown without a lag time. A mixture of chloroform and methanol was added to the culture medium to extract lipids from the medium. As a result, 40-50% of the weight of the dried bacterial cells was composed of lipids.

Also, for production of lipids in a fermentor using mixed volatile fatty acids, 1 L of the culture medium was fed-cultured using a volatile fatty acid mixture of acetic acid:propionic acid:butyric acid (a weight ratio of 6:1:3), which is a composition frequently found in an aerobic digester, while the volatile fatty acid mixture was maintained at a constant concentration by the pH stat method. As a result, it could be seen that OD₆₀₀=32 at 7 days, and 3.2 g/L of lipids was obtained, indicating that 32% of the dried bacterial cells consisted of lipids.

### Example 4

### Production of Microbial Lipids from Volatile Fatty Acids Using Microalgae

The production of lipids from volatile fatty acids was carried out using the microalga Chlorella protothecoides (UTEX 25). To prepare culture media for lipid production, various concentrations of each of acetic acid, propionic acid and butyric acid were added to a modified Endo & Koibuchi medium (containing 0.5 g/L urea, 0.7 g/L KH₂PO₄, 0.3 g/L K₂HPO₄, 0.3 g/L MgSO₄.7H₂O, and 1 ml A5 trace element).

C. protothecoides that had been grown in the presence of 20 g/L glucose was inoculated at a concentration of 10% into the modified Endo & Koibuchi medium and then shake-cultured. As a result, it could be seen that C. protothecoides could effectively use acetic acid, whereas butyric acid could be easily used only at a concentration of 0.5 g/L or less and propionic acid was not substantially used while the growth of the microalgae was severely inhibited.

As a result, in the case of the medium containing 3 g/L acetic acid, C. protothecoides was grown to OD₄₅₀=3.2, and in the case of the medium 2 g/L of a volatile fatty acid mixture having a mixing ratio of acetic acid:propionic acid:butyric acid of 8:1:1, C. protothecoides was grown to OD₄₅₀=1.5. At this time, the content of lipids was in the range of 25-30%.

### Example 5

### Production of Microbial Lipids from Volatile Fatty Acids Using Yeast

Production of lipids from volatile fatty acids was performed using the yeast Cryptococcus albidus (ATCC 10672) in the same medium and culture conditions as Example 1, except for carbon sources.

First, the yeast C. albidus that has been shake-cultured in modified basal medium (containing 30 g/L glucose and 5 g/L NH₄Cl as carbon sources) for 30 hours was inoculated at a concentration of 10% (v/v) into a 250 ml Erlenmeyer flask containing 50 ml of a medium (shown in Table 3 below) and was then cultured under the same conditions.

Table 3 below shows the results of producing microbial lipids from the yeast using mixed volatile fatty acids or volatile fatty acid-derived substances.

TABLE 3

| Carbon | Biomass | Lipid | Lipid Content | Y_{L/S} |
|---|---|---|---|---|
| Source | (g/L) | (g/L) | (%, w/w) | (g/g)^{a} |
| VFAs^{b} | 2.57 | 0.65 | 25.1 | 0.125 |
| Acetic acid | 2.85 | 0.74 | 25.8 | 0.123 |
| Ethyl acetate | 1.38 | 0.26 | 18.5 | 0.058 |
| Sodium acetate | 2.79 | 0.68 | 24.5 | 0.083 |
| Ammonium acetate | 2.39 | 0.58 | 24.1 | 0.075 |
| Calcium acetate | N.D.^{c} | N.D. | N.D. | N.D. |
| Ethanol | 2.11 | 0.53 | 24.9 | 0.114 |

^{a}Y_{L/S}: Lipid yield coefficient, g lipid/g carbon source.
^{b}VFAs ratio was 6:1:3 of acetic acid:propionic acid:butyric acid
^{c}N.D.: not detected

As can be seen in Table 3 above, the mixture of volatile fatty acids (VFAs) showed results similar to lactic acid and acetic acid, indicating that it is suitable for use as carbon sources, but said mixture of volatile fatty acids showed a low lipid content of about 25% and a low yield of about 0.125 g/g.

In order to examine whether acetic acid can be used in a mixture with glucose that is an effective carbon source for microbial growth, production of microbial lipids was performed using various ratios of acetic acid and glucose, and the results are shown in Table 4 below.

TABLE 4

| | Biomass | Lipid | Lipid Content | Y_{L/S} |
|---|---|---|---|---|
| NO. | (g/L) | (g/L) | (%, w/w) | (g/g) |
| #1 | 2.94 | 0.68 | 23.1 | 0.097 |
| #2 | 5.17 | 1.34 | 25.9 | 0.122 |
| #3 | 5.98 | 1.76 | 29.3 | 0.135 |
| #4 | 6.40 | 2.15 | 33.5 | 0.143 |
| #5 | 7.75 | 2.94 | 37.9 | 0.173 |
| #6 | 7.81 | 3.19 | 40.9 | 0.168 |
| #7 | 8.91 | 3.68 | 41.2 | 0.175 |

Carbon sources (g/L): aa; acetic acid, G; glucose.
#1 (7-aa), #2 (5-aa & 6-G), #3 (4-aa & 9-G), #4 (3-aa & 12-G), #5-(2-aa & 15-G), #6-(1-aa & 18-G), and #7 (21-G).
“7-aa” means that 7 g/L acetic acid was used as a carbon source.

As can be seen in Table 4 above, as the concentration of glucose was increased, the yield and content of lipids were increased, but were not greatly different between an acetic acid:glucose ratio of 2:15 (17 g/L) and an acetic acid:glucose ratio of 0:21 (21 g/L). This suggests that a mixture of an inexpensive volatile fatty acid with glucose can be used for the production of microbial lipids.

### Example 6

### Production of Microbial Lipids by 2-Stage Fermentation

In order to establish an effective method for producing microbial lipids, which minimizes microbial growth inhibition resulting from volatile fatty acids, a 2-stage fermentation process was carried out using the lipid-producing yeast Cryptococcus albidus (ATCC 10672). Glucose is a good carbon source for cell growth and lipid accumulation, but is expensive. For this reason, according to the present invention, in the bioreactor for microbial growth, the growth of microbial cells in a medium having a low C/N ratio (1 g/L or less of NH₄Cl) was maximized using a carbon source comprising sugar or glucose, and in the bioreactor for production of microbial intracellular products, the production of lipid in a medium having a high C/N ratio (15 g/L or less of NH₄Cl) was maximized.

The first stage culture and second stage culture of C. albidus (ATCC 10672) were carried out in the modified basal media of Example 1 containing the carbon and nitrogen sources shown in Table 5 below, and the resulting lipid production was measured. The measurement results are shown in Table 5 below.

TABLE 5

| | 1st Stage | 2nd Stage | |
|---|---|---|---|
| Carbon Source | Glucose | VFAs^{a} | Acetic acid |
| Carbon Source Concentration (g/L) | 20 | 9 | 9 |
| NH₄Cl Concentration (g/L) | 5 | 1 | 1 |
| Cultivation Time (h) | 48 | 60 | 60 |
| Residual Carbon Source (g/L) | 4.24 | 0.34 | 1.35 |
| Biomass (g/L) | 5.49 | 7.96 | 8.14 |
| Lipid Concentration (g/L) | 1.18 | 3.49 | 3.12 |
| Lipid Content (%, w/w) | 21.5 | 43.8 | 38.3 |
| Y_{L/S} (g/g) | 0.07 | 0.27 | 0.25 |

^{a}VFAs ratio was 6:1:3 of acetic acid:propionic acid:butyric acid

As can be seen in Table 5 below, when the first stage culture of C. albidus (ATCC 10672) was carried out in the glucose-containing medium, after which the second stage culture was carried out in the VFAs or acetic acid-containing medium, the inhibition of growth of C. albidus (ATCC 10672) by volatile fatty acid was minimized while the same lipid content as that obtained when using glucose was obtained, indicating that the two-stage culture method was highly cost-effective.

Specifically, as can be seen in Table 4 above, when microbial culture was carried out in the medium containing volatile fatty acid from the initial stage, the growth of microbial cells was inhibited and the yield of lipids from the carbon source was also as low as 0.097 g/g. However, when the multi-stage continuous high-cell-density bioreactor was used, the yield of lipids was increased to 0.27 g/g and the content of lipids was 44% or higher, indicating that the yield and content of lipids were similar to those obtained when using glucose.

### Example 7

### Influence of C/N ratio on Lipid Content

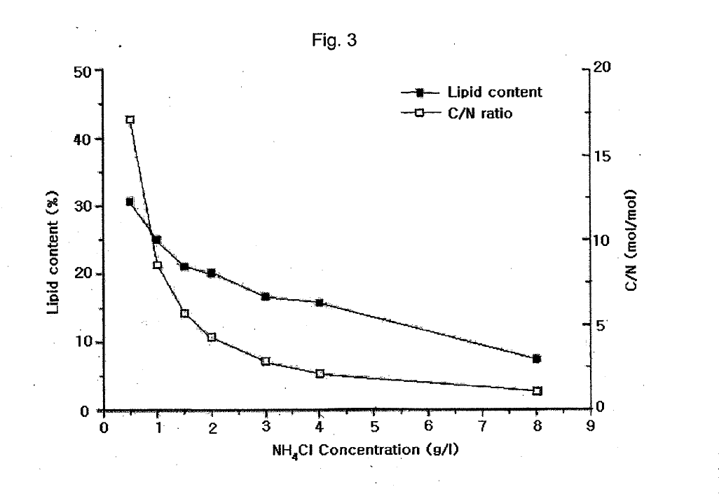

The influence of the C/N ratio on the lipid content was examined using NH₄Cl as a nitrogen source. Cryptococcus albidus (ATCC10672) was cultured in the modified basal medium of Example 1 under the same culture conditions as Example 1 using VFAs as a carbon source and NH₄Cl as a nitrogen source. As a result, as can be seen in FIG. 3, the content of lipids was rapidly increased as the C/N ratio was increased, and in order to provide a lipid content of 30% or higher, the C/N ratio should be 10 or higher.

### Example 8

### Analysis of Composition of Microbial Lipids

Each of Bacteria (R. opacus), microalga (C. protothecoides) and yeast (C. albidus), used in the above Examples, was cultured in each of glucose-containing medium and volatile fatty acid-containing medium, and the composition of lipids in each culture medium was analyzed.

Specifically, the yeast C. albidus was cultured in glucose-containing medium (modified basal medium of Example 1 containing 20 g/L glucose) for 72 hours. For comparison, the yeast C. albidus was cultured in volatile fatty acid-containing medium (modified basal medium of Example 1 containing 9 g/L volatile fatty acid mixture (acetic acid:propionic acid:butyric acid=6:1:3)) for 96 hours.

The microalga C. protothecoides was cultured in glucose-containing medium (medium of Example 4 containing 20 g/L glucose, 0.5 g/L urea, 0.7 g/L KH₂PO₄, 0.3 g/L K₂HPO₄, 0.3 g/L MgSO₄.7H₂O, and 1 ml A5 trace element) for 108 hours. For comparison, the microalga C. protothecoides was cultured in volatile fatty acid-containing medium (medium of Example 4 containing the mixture of volatile fatty acids (acetic acid:propionic acid:butyric acid=6:1:3), 0.5 g/L urea, 0.7 g/L KH₂PO₄, 0.3 g/L K₂HPO₄, 0.3 g/L MgSO₄.7H₂O, and 1 ml A5 trace element) for 168 hours.

The lipids of said bacteria, microalgae and yeasts were extracted with a mixed solvent of chloroform:methanol (1:2) using the Bligh & Dyer method, after which the solvent was evaporated, the methanol and ester of each extract were reacted to form methyl ester, and the composition of each extract was analyzed by GC/MS. As a result, the fatty acids of the bacteria R. opacus had a relatively low content of unsaturated fatty acids, similar to animal fatty acids, and the microalga C. protothecoides showed an unsaturated fatty acid content similar to rapes. Also, the yeast C. albidus showed an unsaturated fatty acid content similar to soybean oil. The content of unsaturated fatty acids in lipids is an important fact that determines the low-temperature flowability of biodiesel converted from the lipids. The higher the content of unsaturated fatty acids, the better is the low-temperature flowability, so that the biodiesel can also be used in cold areas. The fatty acid content of the microorganisms cultured in the glucose-containing medium did not greatly differ from the fatty acid content of the microorganisms cultured in the volatile fatty acid-containing medium, indicating that the production of microbial lipids using volatile fatty acids or volatile fatty acid-containing carbon sources is highly economical.

TABLE 6

| | Carbon | Relative proportion of fatty acids (% w/w) | | | | | | | | | | | | |
|---|---|---|---|---|---|---|---|---|---|---|---|---|---|---|
| Organism | source | C14:0 | C15:0 | C16:0 | C16:1 | C17:0 | C17:1 | C18:0 | C18:1 | C18:2 | C18:3 | C19:0 | C19:1 | C20:0 |
| R. opacus | VFAs | 3.8 | 15.8 | 20.7 | 14.4 | 4.6 | 5.5 | 16.4 | 5.0 | — | — | 6.1 | 5.5 | 2.1 |
| C. | Glucose | 1.2 | — | 14.3 | — | 0.4 | — | 5.4 | 57.6 | 18.6 | 2.5 | — | — | — |
| protothecoides | | | | | | | | | | | | | | |
| | VFAs | — | — | 21.5 | — | — | — | 21.7 | 45.9 | 10.8 | — | — | — | — |
| C. albidus | Glucose | — | — | 17.9 | 0.53 | — | — | 2.88 | 19.6 | 59.1 | — | — | — | — |
| | VFAs | — | — | 16.1 | — | — | — | 5.14 | 17.7 | 61.1 | — | — | — | — |

Myristic acid (C14:0), Pentadecylic acid (C15:0), Palmitic acid (C16:0), Palmitoleic acid (C16:1), Margaric acid (C17:0), Heptadenoic acid (C17:1), Stearic acid (C18:0), Oleic acid (C18:1), Linoleic acid (C18:2), Linolenic acid (C18:3), Nonadecylic acid (C19:0), N/A (C19:1), and Arachidic acid (C20:0)

As can be seen in Table 6 above, even when volatile fatty acids (VFAs) were used instead of glucose as a carbon source, the productivity, composition and the like of produced lipids (fatty acids) vary depending on the kind of cultured microorganisms, but do not greatly differ from those obtained when using glucose. In addition, because the composition of lipids can be changed depending on the kind of cultured microorganisms, various lipids can be selectively produced.

### Example 9

### Production of Microbial Lipids Using Multi-Stage Continuous High-Cell-Density Bioreactor

In general biological processes, raw material price, conversion rate, production yield, concentration, and reactor's productivity play important roles. As confirmed in Examples 1 to 8 above, the use of VFAs can greatly reduce the raw material price, and the application of multi-stage culture can increase the concentration of the products. However, in order to increase the production of lipids, each reactor is preferably composed of a multi-stage continuous high-cell-density culture (MSC-HCDC) system.

The continuous high-cell-density reactor is composed of a two-stage reactor system, in which the first reactor can be operated focusing on the production of microbial cells, like a fed-culture process, and the second reactor can be operated focusing on the accumulation of intracellular products in microbial cells.

In the second reactor system, it is very important to determine whether the reactor is operated in the form of a continuous stirred tank reactor (CSTR) or operated in the form of PFR (plug flow reactor). For example, given a first-order reaction, the consumption rate of lipid which is an intracellular product is given as C₁=C₀/(1+k₁Θ_{T}) for CSTR, Cₙ=C₀/(1+k₁.Θ_{T}/n)ⁿ for several reactors, and C₁=C₀exp(−k₁.Θ_{T}) for the PFR, wherein Θ_{T} is total residence time, k₁ is rate constant, C₀ is initial concentration, and Cₙ is the lipid concentration of the n^{st} reactor. The higher the Cₙ value, the higher is the rate of conversion to lipids, and the higher is lipid concentration (quoted from a chemical reaction engineering textbook).

Where several CSTRs are used, when conversion rate is 90% (C₁=0.1C₀), the theoretically predicted values of lipid productivity are 208% for n=2, 260% for n=3, and 390% for the PFR, if the lipid productivity of n=1 is assumed to be 100%. The lipid productivity increases as the rate of conversion to lipids and the number of reactors increase. The maximum value of the lipid productivity converges to the value of the PFR.

Another method for increasing lipid productivity is to change the lipid-producing strain from C. albidus to E. coli having a higher growth rate. This effort has been conducted by the Keasling group at Berkeley university (Steen, E. J. et al, Nature, 463, pp 559-562, 2010), and it was reported that 0.2 g/(L.h) of lipids per g microbial cells can be obtained in a commercial process.

In summary, the productivity of the bacterial strain itself and the reactor efficiency determine the total productivity of the reactor. Table 7 below shows the efficiency of the reactor system as a function of the number of lipid-producing reactors and the rate of conversion to lipids, determined under the condition in which the total residence time in the reactor was fixed. If the results of Table 7 are realized, the production cost of lipids can be lowered to 0.44 $/kg or less. All the results of Table 7 are attributable to high production costs, and thus if a lipid-producing factory is constructed in a country in which lipids are produced at low costs, the production of lipid can become more cost-effective.

TABLE 7
Lipid productivity (g/L/h)

| | Microbial cells | Con- | | | | |
|---|---|---|---|---|---|---|
| | growth rate (K₁), | ver- | | | | |
| | lipid production | sion | | | | |
| system | rate (K₂) | rate | 1-CSTR | 2-CSTR | 3-CSTR | PFR |
| system 1 | k₁ = 0.1, k₂ = 0.1 | 90% | 5 | 10.4 | 13.0 | 19.5 |
| system 2 | k₁ = 0.2, k₂ = 0.1 | 90% | 6 | 12.4 | 15.6 | 23.4 |
| system 3 | k₁ = 0.4, k₂ = 0.2 | 90% | 12 | 24.9 | 31.2 | 46.8 |
| system 4 | k₁ = 0.8, k₂ = 0.4 | 90% | 24 | 49.2 | 62.4 | 93.6 |
| system 2-1 | k₁ = 0.2, k₂ = 0.1 | 50% | 54 | 64.8 | 69.12 | 77.76 |
| system 2-2 | k₁ = 0.2, k₂ = 0.1 | 95% | 2.82 | 7.614 | 10.35 | 17.77 |

Description of the systems:
R₁ (for production of microbial cells), and
R₂ (for production of lipids);
a plurality of R₂ reactors are connected with each other to form several CSTRs to increase reaction efficiency (total residence time Θ_{T} is constant;
K₁ = cell growth rate (1/h); and
K₂ = lipid production rate (1/h)).

In Table 7 above, system 1 has a configuration in which two R₂ reactors (for production of lipids) are connected to one R₁ reactor (for production of microbial cells), and systems 2 to 4 have a configuration in which 4 R₂ reactors (for production of lipids) are connected to one R₁ reactor (for production of microbial cells).

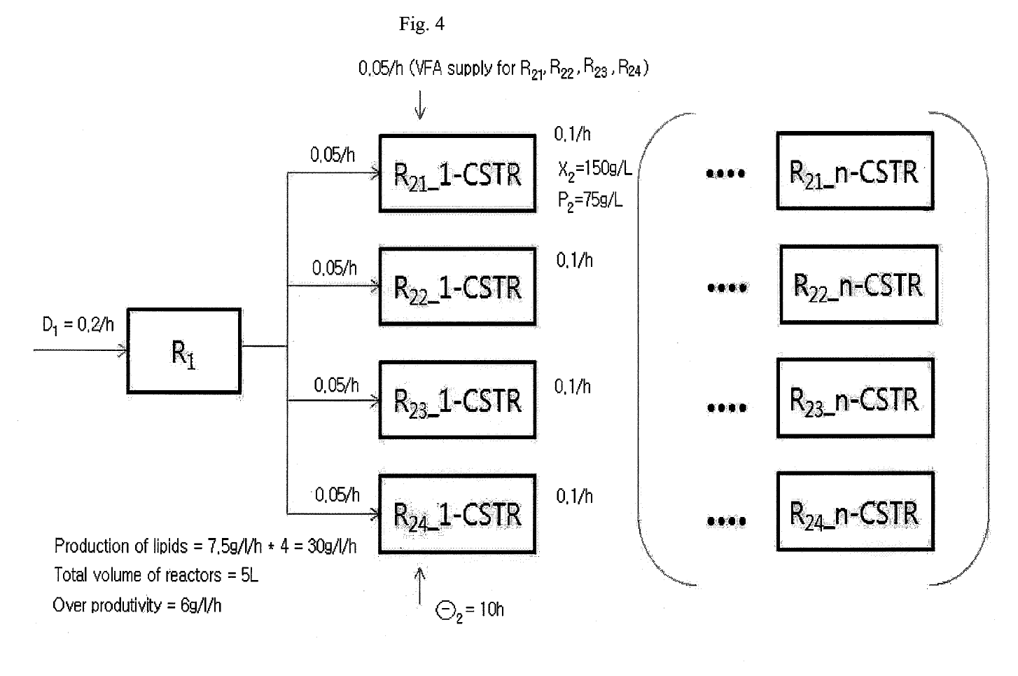

The conversion rate in Table 7 indicates the rate of conversion from volatile fatty acids to lipids, and 1-CSTR, 2-CSTR and 3-CSTR are indicated according to the number of R₂ reactors (for production of lipids) connected in series to each other. As shown in FIG. 4, the configuration of up to n-CSTR is possible.

In Table 7, system 2-1 and system 2-2 indicate the cases in which the conversion rates in system 2 were changed 50% and 95%, respectively.

FIG. 4 shows multi-stage continuous high-cell-density bioreactor system 2. As can be seen therein, the reactor for production of microbial cells has a growth rate of 0.2/h that is the same value of dilution rate a dilution rate (D₁), and D₁ is divided into four equal parts (each part is 0.05/h) which are then introduced into four R₂ reactors (for production of lipids). The volatile fatty acid solution for production of lipids is introduced into each of the four reactors at a flow rate of 0.05/h, and then each of the reactors discharges a lipid solution at a flow rate of 0.1/h.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method of producing microbial intracellular products, the method including growing microorganisms in a bioreactor and subjecting the grown microorganisms to fed-batch culture using volatile fatty acids as substrates, thereby producing microbial intracellular products.

2. The method of claim 1, said volatile fatty acids are selected from the group consisting of acetic acid, propionic acid, butyric acid, lactic acid, succinic acid, gluconic acid, valeric acid, caproic acid, and mixtures thereof.

3. The method of claim 1, said microorganisms are selected from the group consisting of bacteria, yeasts, fungi and microalgae.

4. A method of producing microbial intracellular products from volatile fatty acids in a multi-stage continuous high-cell-density bioreactor system, the method including the steps of: (a) culturing microorganisms in a bioreactor for microbial growth, thereby growing the microorganisms; (b) culturing the grown microorganisms of step (a) in a bioreactor for production of microbial intracellular products, which includes a medium containing volatile fatty acids, thereby producing microbial intracellular products; and (c) recovering the microbial intracellular products from the culture medium of the bioreactor for production of microbial intracellular products.

5. The method of claim 4, each of the bioreactor for microbial growth and the bioreactor for production of microbial intracellular products consists of a reactor for multi-stage continuous high-cell-density culture (MSC-HCDC), and the bioreactor for microbial growth is operated in the form of a continuous stirred tank reactor (CSTR), and the bioreactor for production of microbial intracellular products is operated in the form of several CSTRs or is operated in the form of a plug flow reactor (PFR) provided by dividing the inside of one CSTR into several sections.

6. The method of claim 4, said bioreactor for microbial growth is for growing of microorganism, and said bioreactor for production of microbial intracellular products is for producing of microbial intracellular products by fed-batch culture using volatile fatty acids as substrates.

7. The method of claim 4, said method further includes the step of producing volatile fatty acids from organic waste or inexpensive organic materials before said step (a) or said step (b).

8. The method of claim 7, said organic waste is selected from the group consisting urban waste, food waste, sludge, livestock excretions and organic biomass

9. The method of claim 4, said volatile fatty acids are selected from the group consisting acetic acid, propionic acid, butyric acid, lactic acid, succinic acid, gluconic acid, valeric acid, caproic acid, and mixtures thereof.

10. The method of claim 4, said microorganism is selected from the group consisting of bacteria, yeasts, fungi and microalgae.

11. The method of claim 4, said medium of microorganisms in the bioreactor for microbial growth contains (1) a carbon (C) source selected from the group consisting of glucose, xylose, arabinose, sugar, alcohol, sugar alcohol, a saccharification product of biomass, and mixtures thereof, and (2) a nitrogen (N) source selected from the group consisting of ammonia-derived compounds, nitric oxide, urea, corn steep liquor, food waste, and mixtures thereof.

12. The method of claim 4, said volatile fatty acid-containing medium which is included in the bioreactor for production of microbial intracellular products contains, in addition to the volatile fatty acids, (1) a carbon (C) source selected from the group consisting of glucose, xylose, arabinose, sugar, alcohol, sugar alcohol, a saccharification product of biomass, and mixtures thereof, and (2) a nitrogen (N) source consisting of ammonia-derived compounds, nitric oxides, urea, corn steep liquor, food waste, and mixtures thereof.

13. The method of claim 4, said volatile fatty acids is maintained at a volatile fatty acid concentration of 1-5 g/L during culture in the bioreactor.

14. The method of claim 4, the remaining microorganisms after recovering the microbial intracellular products from the culture medium of the bioreactor for production of microbial intracellular products are used as a microbial strain for production of volatile fatty acids.

15. The method of claim 4, the microbial intracellular products are selected from the group consisting of intracellular proteins, intracellular organic polymers, and microbial lipids.
